(19) United States
(12) Patent Application Publication
(10) Pub. No.: US 2008/0052010 A1
(43) Pub. Date: Feb. 28, 2008

(54) Method and Apparatus for Analyzing Signal Pattern of Sensor Array

(75) Inventors: Seung Lee, Gangwon-do (KR); Chang-Hwa Kim, Gangwon-do (KR); Sang-Kyung Kim, Gangwon-do (KR); A-Ram Kim, Gangwon-do (KR)

Correspondence Address: WELSH & KATZ, LTD, 120 S RIVERSIDE PLAZA, 22ND FLOOR, CHICAGO, IL, 60606, US

(73) Assignee: Kangnung National University Industry Academy Corporation Group, Gangwon-do (KR)

(21) Appl. No.: 11/778,729

(22) Filed: Jul. 17, 2007

(30) Foreign Application Priority Data
Aug. 22, 2006 (KR) 10-2006-0079597

(51) Int. Cl. G01N 33/00; G06F 19/00

(52) U.S. Cl. 702/24

(57) ABSTRACT

An aspect of the present invention features a method for analyzing a signal pattern detected by a sensor array that comprises one or more gas sensors. The method can comprises converting multidimensional data outputted from the sensor array to linear data, the data containing information on one or more reference gases; creating an ADSTM (Angle Difference-based State Transition Model) by using the converted data; and analyzing a gas by using the ADSTM when the sensor array outputs data of the gas. The method for analyzing a signal pattern detected by a sensor array according to the present invention can convert multidimensional values inputted through the sensor array into a single piece of continous data.

### CROSS-REFERENCE TO RELATED APPLICATIONS

This application claims the benefit of Korean Patent Application No. 2006-0079597 filed with the Korean Intellectual Property Office on Aug. 22. 2006, the disclosures of which are incorporated herein by reference in their entirety.

### BACKGROUND OF THE INVENTION

1. Technical Field

The present invention relates to a method and an apparatus for analyzing a signal pattern of a sensor array and, more particularly, to a method and an apparatus that can convert a signal pattern detected by a sensor array into desired information through modeling.

2. Description of the Related Art

Apparatuses with a sensor are being widely used in most fields of industries. Depending on its function, the sensor is called an optical sensor, a temperature sensor, a gas sensor, a humidity sensor, an acoustic sensor, etc.

Furthermore, the sensors are expected to perform many functions in from a conventional application field such as a measurement system to a robotic field, a medical field, a safety and security field, and a ubiquitous field.

In order to fully function, it is required of the sensor not only to sense a change in the external environment but also to be capable of analyzing or converting measured values into a desired form.

Here, the method by which the sensor senses may be different depending on the information, which can be physical information (for example, distance, thickness, weight, etc.), electrical information (for example, current, voltage, registance, etc.), chemical information (density, purity, etc.), bio information, etc.

In order to convert the measured value into a desired form of information, meaning assignment is required. For example, a gas leakage detector is required to raise an alarm when it realizes that gas is leaking more than a reference value. That is, the value inputted to the gas detector plays its role by allowing the gas leakage detector to sound an alarm.

Meanwhile, converting a signal into information includes converting data into a pattern and assigning a meaning thereto.

Generally, a sensor array is formed with a plurality of channels, through which a large amount of data is supplied. Therefore, the sensor array employs a multivariate analysis method, which is roughly devided into a statistical method and a neural network method. The analysis method is chosen based on properties of data and analysis environment.

The following introduces typical methods used by a gas sensor to recognize a signal pattern. Here, the pattern recognition includes outputting signal in a gas sensor and obtaining result using the signal, to which the pattern recognition methods like above are applied.

To the pattern recognition was first applied the statistical method. And then, with appearance of a nueral network theory, developments associated with the neural network were achieved, introducing a recognition process imitating human recognition process, a composite recognition process incorporating the neural network with the statistical method, etc.

A signal pattern recognition method for a gas sensor array has similar process with a general pattern recognition method, so that it includes extracting a gas signal pattern form gas data, analyzing the gas signal pattern and classifying the gas signal pattern or measuring similarity.

A method of analyzing data inputted from a plurality of sensors is devided into a statistical method and a neural network method. The statistical method classifies a pattern based on a statistical model. The statistical model classifies and recognizes the pattern based on Bayer's rule.

The neural network method, imitating human neural network process, inputs a pattern from the sensor as a single input value, and uses a network composed of processing units (neurons) to process the inputted pattern. In this case, the information on the pattern is stored as weight of synapses, which allow the network to be connected.

The neural network methd can adjust the weight through learning, undergoes no standardized process such as an algorithm, and can handle as a black box a process from input to output in the neural network.

And, the neural network method rarely needs preliminary information, can create theoritically any complicated determination region if the neuron network has enough neuron layers. Also, the neural network method is highly resistive to noise.

The neural network method can eliminate noise through learning to focus on analysis of the original data.

Referring to Table 1, a table where algorithms for recognizing a gas signal pattern are classified, the statistical method and the nueral network method are devided into a supervised method and an unsupervised method.

TABLE 1
Gas Signal Pattern Recognition Algorithm

| | | |
|---|---|---|
| Statistical | Unsupervised | Cluster Analysis(CA) |
| | | Principal Component Analysis(PCA) |
| | Supervised | Discriminant Function |
| | | Analysis(DFA) |
| | | Template Matching |
| Neural Network | Unsupervised | Self-Organized Map(SOM) |
| | | Genetic Algorithm(GA) |
| | | Adaptive Resonance Theory(ART) |
| | Supervised | Learning Vector Quantization(LVQ) |
| | | Back Propagation(BP) |
| | | Radial Basis Function(RBF) |
| | | Fuzzy Learning Vector |
| | | Quantization(FLVQ) |

The supervised method specifies a measuring range in advance, and determines whether or not a measured value is within the the range. And, the unsupervised method identifies gases, without preliminary information, through learning the measured values.

In other words, the supervised method has preliminary information on a target gas and uses the information as a reference to measured values. However, the unsupervised method performs a number of measurements, and creates a reference through learning.

Especially, the nueral network method can process nonlinear data, and has high resistance to deviation or noise, so that it is mainly used in the field of gas recognition.

But, the neural network is poor at learning temporal corelation. Furthermore, when the unsupervised method is employed, it takes long learning time, and unlearned data hardly is identified.

Also, the conventional algorithm does not reflect features of the signal pattern or takes long time to learn a gas model.

### SUMMARY OF THE INVENTION

The present invention provides a method and an apparatus for analyzing a signal pattern of a sensor array that can convert multidimensional values inputted through the sensor array into a single piece of continous data.

Furthermore, the present invention provides a method and an apparatus for analyzing a signal pattern of a sensor array that can extract the properties of the converted data to perform modeling of a signal pattern detected by a sensor.

Also, the present invention provides a method and an apparatus for analyzing a signal pattern of a sensor array that can improve the analysis by arranging components of a model appropriately.

An aspect of the present invention features a method for analyzing a signal pattern detected by a sensor array that comprises one or more gas sensors. The method can comprises converting multidimensional data outputted from the sensor array to linear data, the data containing information on one or more reference gases; creating an ADSTM (Angle Difference-based State Transition Model) by using the converted data; and analyzing a gas by using the ADSTM when the sensor array outputs data of the gas.

The method can further comprises preprocessing the multidimensional data; extracting a meaning section from the preprocessed data; linearizing data contained in the meaning section; extracting a k numbe of samples from the linearized data (k is a natural number); and quantizing the data samples.

The method can further comprises generating a plurality of transition vectors using the quantized data; dividing a predetermined section into an n number of sections, each section defined as one state; generating a state sequence by corresponding angles of the transition vectors with the n number of sections; and generating for each reference gas one or more first state transition matrices to correspond to the number of transitions by using the state sequence.

Another aspect of the present invention features a recorded medium readable by a computer, tangibly embodying a program of instructions executable by the computer to perform the method.

Another aspect of the present invention features an apparatus for analyzing a signal pattern detected by a sensor array that comprising one or more gas sensors. The apparatus can comprise a preprocessor that preprocesses multidimensional data outputted from the sensor array; a meaning-section extractor that extracts a meaning section from the preprocessed data; a linearizing part that linearizes the multisimensional data contained in the meaning section; a quantizing part that extracts a k number of samples (k is a natural number) from the linearized data and quantizes the samples; a model creating part that creates an ADSTM(Angle Difference-based State Transition Model) by using the quantized data; and an analyzing part that analyzes a gas by using the ADSTM when the sensor array outputs data of the gas.

The apparatus can further comprises a transition vector generating part that generates a plurality of transition vectors by using the quantized data; a section dividing part that divides a predetermined section into an n number of sections, each section defined as one state; a state sequence generating part that generates a state sequence by corresponding angles of the transition vector with the n number of sections; and a state transition matrix generating part that generates for each reference gas one or more first state transition matrices to correspond to the number of transitions by using the state sequence.

Additional aspects and advantages of the present general inventive concept will be set forth in part in the description which follows, and in part will be apparent from the description, or may be learned by practice of the general inventive concept.

### BRIEF DESCRIPTION OF THE DRAWINGS

These and other features, aspects, and advantages of the present invention will become better understood with regard to the following description, appended claims, and accompanying drawings where:

FIG. 1 is a block diagram of a signal pattern analysis apparatus according to an aspect of the present invention.

FIG. 2 is a graph showing a signal pattern outputted by a sensor array.

FIG. 3 is a graph showing a meaning section in a signal pattern outputted by a sensor array according to an aspect of the present invention.

FIG. 4 illustrates a meaning section extracted from the graph in FIG. 3.

FIG. 5 is a graph of a linearized gas signal pattern according to an aspect of the present invention.

FIG. 6 is a graph of a linearized gas signal pattern quantized

FIG. 7 illustrates states classified according to an aspect of the present invention.

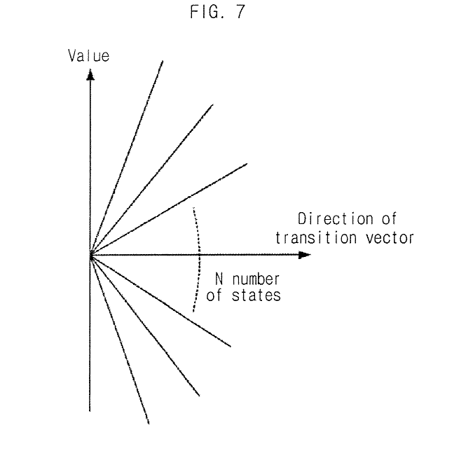

FIG. 8 is the result of extracting samples from measured values of toluene gas.

FIG. 9 is a state transition diagram according to an aspect of the present invention.

FIG. 10 is a flow chart of a signal pattern analysis according to an aspect of the present invention.

FIG. 11 shows the results of similarity measurements for toluene gas, with a fixed sampling frequency, according to an aspect of the present invention.

FIG. 12 shows the results of similarity measurements for benzene gas, with a fixed sampling frequency, according to an aspect of the present invention.

FIG. 13 shows the results of similarity measurements for cyclohexane gas, with a fixed sampling frequency, according to an aspect of the present invention.

FIG. 14 shows the results of similarity measurements for ethanol gas, with a fixed sampling frequency, according to an aspect of the present invention.

FIG. 15 shows the results of similarity measurements for heptane gas, with a fixed sampling frequency, according to an aspect of the present invention.

FIG. 16 shows the results of similarity measurements for hexane gas, with a fixed sampling frequency, according to an aspect of the present invention.

FIG. 17 shows the results of similarity measurements for methanol gas, with a fixed sampling frequency, according to an aspect of the present invention.

FIG. 18 shows the results of similarity measurements for propanol gas, with a fixed sampling frequency, according to an aspect of the present invention.

FIG. 19 shows the results of similarity measurements for toluene gas, with a fixed number of states, according to an aspect of the present invention.

FIG. 20 shows the results of similarity measurements for benzene gas, with a fixed number of states, according to an aspect of the present invention.

FIG. 21 shows the results of similarity measurements for cyclohexane gas, with a fixed number of states, according to an aspect of the present invention.

FIG. 22 shows the results of similarity measurements for ethanol gas, with a fixed number of states, according to an aspect of the present invention.

FIG. 23 shows the results of similarity measurements for heptane gas, with a fixed number of states, according to an aspect of the present invention.

FIG. 24 shows the results of similarity measurements for hexane gas, with a fixed number of states, according to an aspect of the present invention.

FIG. 25 shows the results of similarity measurements for methanol gas, with a fixed number of states, according to an aspect of the present invention.

FIG. 26 shows the results of similarity measurements for propanol gas, with a fixed number of states, according to an aspect of the present invention.

FIG. 27 shows the result of similarity measurements after applying a threshold value according to an aspect of the present invention.

### DETAILED DESCRIPTION OF THE INVENTION

The following is a detailed description and explanation of the preferred embodiments and best modes contemplated by the inventors of carrying out the invention along with some examples thereof.

Hereinafter, embodiments of the invention will be described in more detail with reference to the accompanying drawings. In the description with reference to the accompanying drawings, those components are rendered the same reference number that are the same or are in correspondence regardless of the figure number, and redundant explanations are omitted.

The present invention relates to a signal pattern analysis of a multi-channel sensor array. Although the description below describes a signal pattern analysis of a gas sensor array, it should be noted that such a description is not intended to limit the scope of the invention.

The present invention uses a state transition model for modeling, and analyzes a created model with a statistical method.

Multidimensional signals inputted from a sensor array are transformed into a single set of continuous data, and a gas signal pattern is drawn from the transformed data to be modeled.

In the gas signal pattern recognition method, data of a target gas is preprocessed to be stored, so that new data of an input gas can be compared with the stored data value.

In the case of using the state transition model, how the properties of data are reflected affects the effectivness of the gas signal pattern recognition. Therefore, the components of the model should be arranged appropriately to increase the effectiveness of the analaysis.

The state transition model is composed of a number of states, which are related to each other by a temporal deductive relation, spatial positional relation, or input/output relation.

The relation between the states, as a continuous relation, represents features or direction of the transition.

The gas sensor is more required for recognizing a toxic gas than an innocuous gas.

Also, in some cases, promptness may be a key factor in the gas signal pattern recognition. And, the state transition modeling and the analyzing method of the present invention uses a statistical method to analyze the gas signal pattern, thereby analyzing a gas signal pattern promptly.

The time spent for identifying the gas signal pattern depends on the time spent for modeling and analyzing the gas signal pattern. It takes an O(N) of time to perform modeling, and an O(N²) of time to analyze. Here, the N is the number of the states.

According to the present invention, the N is defined as a doubledigit number or a singledigit number, so that the overall process takes short time.

Also, the amount of input data does not affect on the accuracy of the analysis so that the method using the state transition model is more advantageous in accuracy and speed than the neural network method that needs more calculation time as the amount of the data increases.

According to the present invention, a method called ADSTM (Angle Differnce based State Transition Modeling) is introduced, which quantitizes a measured gas signal pattern, and then applies transition relation of angles to modeling.

In order to identify the ingredients of a gas is calculated a similarity between the stored data and newly inputted data.

In the present invention, the similarity is calculated through measuring a spatial similarity between state transition matrices generated by the state transition modeling method.

In the present invention, the signal outputted by the sensor array has multidimensional data measured by a plurality of channels of the sensor array.

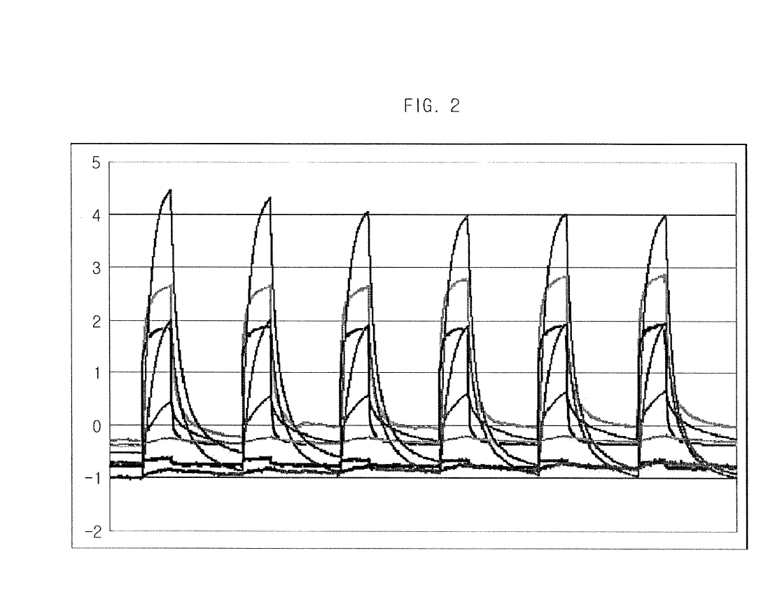

FIG. 2 is a graph, created by repeating measurements until the gas is recognized, shows change of values measured by each sensor according to the progress of time.

In FIG. 2, the data has a multidimensional form that is composed of eight two-dimensional graphs.

In general, a relatively simple method is employed to calculate the similarity between common functions or two-dimensional curves. The simplest method for calculating the similarity is to compare by measuring distance between the curves.

And, among the distance measuring methods is widely used a method using the Euclidean space, which compares the distance between each pair of points constituting the curve.

Since data on different components is inputted to each different channel of the sensor array, when measuring two different gases, each two signals measured by the majority of the channels may have similar patterns except by one or two channels.

In such a case, although two gas signal patterns look similar and have a very small value of Euclidean space distance, the two gases should be distinguished because they have different organizions of components.

However, there are possibilities that different gases are mistakenly regonized as the same gas when using the Euclidean distance measurement method.

Thus, for an accurate analysis, the most important is to fully represent characteristics of each gas curve and to model a gas signal pattern easy to identify.

The ADSTM should extract elements satisfying a modeling reference from a sensor array signal as shown in FIG. 2.

And, the state transition modeling is applied in analyzing the sensor array signal so that the characteristic and the meaning of signal is reflected on the modeling.

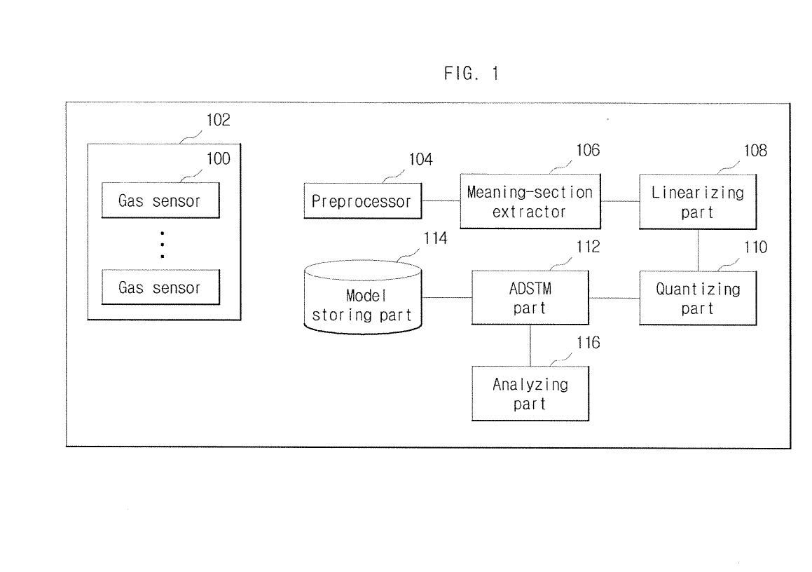

FIG. 1 is a block diagram of a signal pattern analysis apparatus according to an embodiment of the present invention.

Referring to FIG. 1, the signal pattern analysis apparatus may include a sensor array 102 having one or more sensors 100, a preprocessor 104, a meaning-section extractor 106, a linearizing part 108, a quantizing part 110, an ADSTM part 112, a model storing part 114 and an analyzing part 116.

Here, the following description focuses on a case where the sensor array 102 is composed of many kinds of gas sensors.

The gas sensor 100 detects reaction heat or a change in electric conductivity, and converts it to an electric signal. The gas sensor 100 includes a semiconductor type, catalytic combustion type, etc.

The preprocessor 104 extracts elements for modeling from values outputted by the sensor array 102. The values detected by the sensor array are measured repeatedly, so that the values are repeatedly inputted into the preprocessor 104, creating a pattern as shown in FIG. 2.

In order to generate a model, the preprocessor 104 forms a pattern composed of the average of the measured values repeatedly inputted. Here, the measured values that are highly deviated from normal measured values are excluded when calculating the average. Especially, in most cases, the initial value is erroneous. The preprocessor 104 excludes such an initial value of the gas signal pattern to calculate the average.

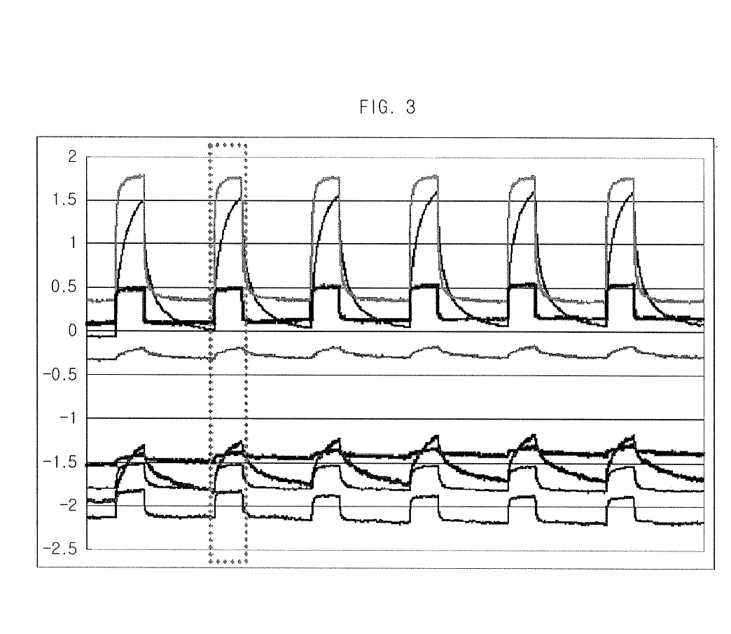

Referring to FIG. 2 and FIG. 3, it can be seen that the values measured by the sensor array 102 repeat rising and dropping. Here, the rising portion indicates a process in which a gas increases to reach a peak, and the dropping portion indicates a process in which the gas decreases to return to a state where no gas is detected.

Accordingly, the meaning section extractor 106 extracts the rising portion as a meaning section. That means, the meaning section extractor 106 continuously monitors values measured by the sensor array 102, and extracts the measured values from when the value begins to increase until when the value reaches to a peak.

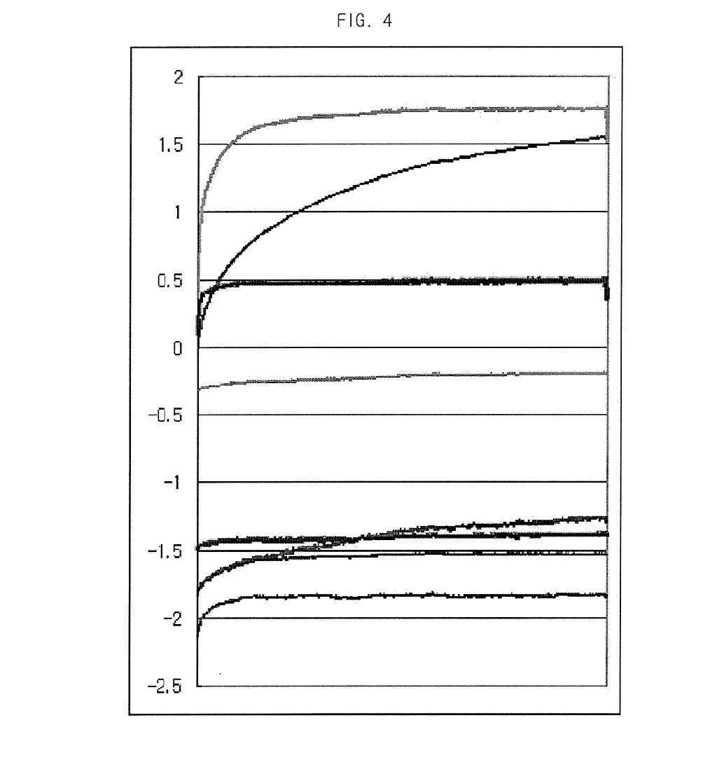

The meaning section extracted may be illustrated as shown in FIG. 4.

For modeling, the linearizing part 108 converts multidimensional data extracted from the meaning section into linear data. The linearizing part 108 also removes noise in order to enhance the reliability of model.

Multidimensional data of the meaning section is converted into one-dimensional linear data by arranging it in a linear form, thereby facilitating the modeling process. During the converting process, all the channels should be arranged in a fixed order.

The quantizing part 110 classifies the converted data into different states in order to apply to the state transition model, and quantizes the data in order to obtain transition relationships between the states.

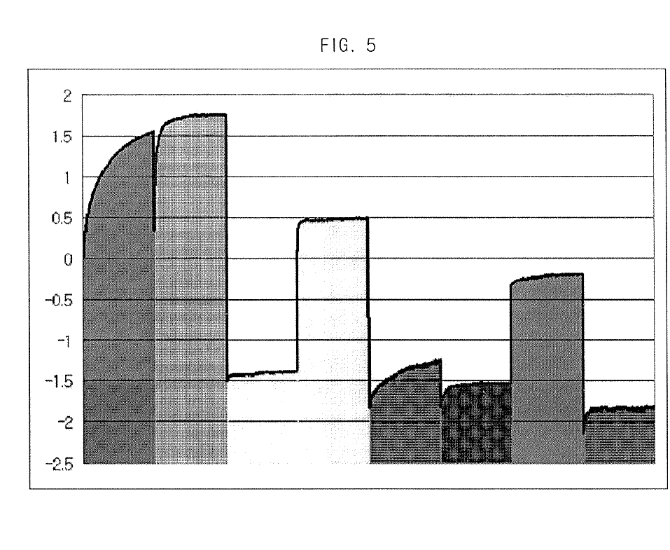

The data is measured at a very short interval so that it forms a curved line as shown in FIG. 5 when arranged.

The quantization includes selecting data at an equal interval larger than the measuring interval, and connecting the selected data to form a broken line graph.

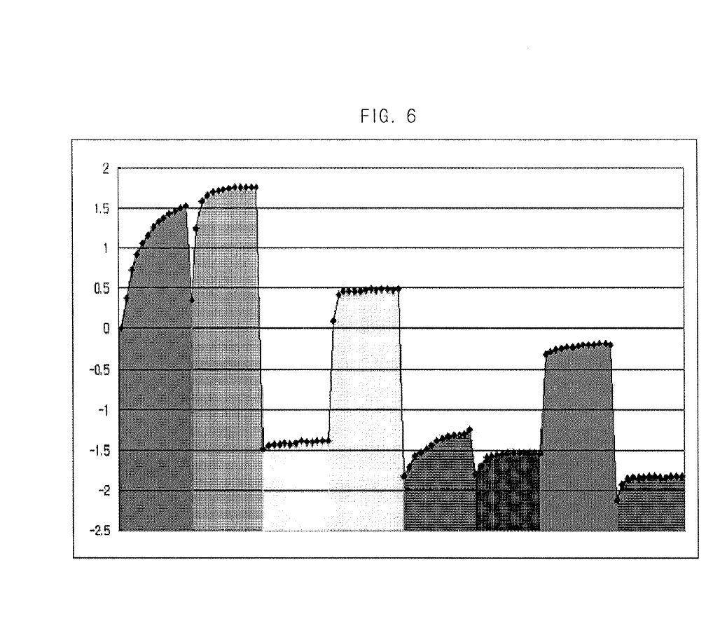

In FIG. 6, sampling is performed k number of times for each channel. With the pattern quantization, a Markov relationship between the quantized elements can be known. In the case that the sensor array has a single sensor, the total number of the quantized elements can be calculated by the following Mathematical Formula 1:

m=k×1 Mathmatical Formula 1

Here, m is the number of the quatized elements, and the k is the sampling frequency.

As a result of the quantization, an m numbe of points are generated, and each distance between adjacent points along the x axis is the same since the sampling is performed at an equal interval. Assuming that the distance along the x axis is 1, a slope between adjacent points can be obtained by knowing a variation along the y axis.

The quantized i th value is represented as qᵢ.

Although not shown in figures, the ADSTM part 112 may include a transition vector generating unit that generates a plurality of transition vectors by using the quantized data, a section division part that divides a predetermined portion into an n number of sections-each section is defined as one state-, a state array generating part that generates a state array by corresponding the transition vectors with the n number of sections based on angle of the transition vector, and a state transition matrix generating part that generates one or more state transition matrices to correspond to the number of transitions for a reference gas by using the state array.

First, the ADSTM part 112 creates a state transition model by using the relationship between adjacent measured values as a transition factor. The ADSTM is basically composed of states and transition between the states.

Here, a transition vector in a direction from qi to qi+1 is represented as q̅ᵢ̅q̅ᵢ̅+̅1̅. Accordingly, when there are an m number of points, an (m−1) number of transition vectors can be obtained as q̅₁̅q̅₂, q̅₂̅q̅₃, . . . , q̅ₘ̅₋̅₁̅q̅ₘ.

Also, the angle of the transition vector refers to an angle that the transition vector forms with respect to the x axis.

In the present invention, when creating a model from the sensor array signals, the direction of the transition vector is used as a state of a state transition model.

Here, since all the transition vectors head for a positive direction with respect to the x axis, the transition vector exists in the quadrant I or IV, thereby having an angle between

$$- \frac{π}{2} and \frac{π}{2} .$$

Here, the range between

$$- \frac{π}{2} and \frac{π}{2}$$

is divided into n sections having an equal size, and each section is defined as a state. In order to simplify calculation, the n is selected among even numbers.

The i th state, sᵢ, is positioned of which range can be represented by the following Mathmatical Formula 2.

$$\begin{matrix}\frac{π}{n} \times ( i - 1 ) \leq {s}_{i} < \frac{π}{n} \times i & ( 0 < i \leq \frac{n}{2} ) \\ - \frac{π}{n} \times ( i - \frac{n}{2} ) \leq {s}_{i} < - \frac{π}{n} ( i - \frac{n}{2} + 1 ) & ( \frac{n}{2} < i \leq n )\end{matrix} \qquad Mathematical Formula 2$$

Here, when

$$0 < i \leq \frac{n}{2} ,$$

the transition vector has a positive angle and when

$$\frac{n}{2} < i \leq n ,$$

the transition vector has a negative angle.

The angle of the transition vector can be obtained from y value of the transition vector by using an arctangent function, when the x value of the transition vector is normalized to 1.

Therefore, the angle dᵢ of the transition vector in the ith state can be computed by the Mathmatical Formula 3.

dᵢ=arc tan(qᵢ−qᵢ₋₁) (1≦i≦m−1) Mathmatical Formula 3

A state transition model for identifying the gas signal pattern is created by using a sequence of the transition vector angles computed as the above.

The state transition model is stored in the model storing part 114.

TABLE 2
State Slope Range

| State number | Range |
|---|---|
| 1 | $0 \leq {S}_{1} < \frac{π}{n}$ |
| 2 | $\frac{π}{n} \leq {S}_{2} < \frac{2 π}{n}$ |
| ... | ... |
| $\frac{n}{2}$ | $\frac{π}{2} - \frac{π}{n} \leq {S}_{\frac{n}{2}} < \frac{π}{2}$ |
| $\frac{n}{2} + 1$ | $- \frac{π}{n} \leq {S}_{\frac{n}{2} + 1} < 0$ |
| $\frac{n}{2} + 2$ | $- \frac{2 π}{n} \leq {S}_{\frac{n}{2} + 2} < - \frac{π}{n}$ |
| ... | ... |
| n | $- \frac{π}{2} \leq {S}_{n} < - \frac{π}{2} + \frac{π}{n}$ |

The range from

$$- \frac{π}{2} to \frac{π}{2}$$

is divided into the n number of states, and the transition vector angle in each state can be sequenced as Table 2.

This sequence of the transition vector angles can be converted into an n×n state transition matrix. When a transition from sᵢ to sⱼ occurs, aᵢⱼ(1≦i≦n, 1≦j≦n) is obtained, so that a matrix can be created as shown in Table 3.

TABLE 3
State Transiton Matrix

| | j [symbol] | | | | |
|---|---|---|---|---|---|
| i [symbol] | S₁ [symbol] | S₂ [symbol] | S₃ [symbol] | . . . [symbol] | Sₙ [symbol] |
| S₁ [symbol] | a₁₁ [symbol] | a₁₂ [symbol] | a₁₃ [symbol] | . . . [symbol] | a₁ₙ [symbol] |
| S₂ [symbol] | a₂₁ [symbol] | a₂₂ [symbol] | a₂₃ [symbol] | . . . [symbol] | a₂ₙ [symbol] |
| S₃ [symbol] | a₃₁ [symbol] | a₃₂ [symbol] | a₃₈ [symbol] | . . . [symbol] | a_{Sn} [symbol] |
| . [symbol] | . [symbol] | . [symbol] | . [symbol] | . [symbol] | . [symbol] |
| . | . | . | . | . | . |
| . | . | . | . | . | . |
| Sₙ [symbol] | aₙ₁ [symbol] | aₙ₂ [symbol] | aₙ₈ [symbol] | . . . [symbol] | aₙₙ [symbol] |

After the state transition model is created as described above, the analyzing part 116 receives the measured values from the sensor array 102, and analyzes the kind and the density of the detected gas.

Hereinafter, process of applying the ADSTM to the sensor array 102 will be described in detail.

By using the above described modeling method, modeling is performed on data actually measured by the sensor array.

Table 4 shows an example of modeling of a toluene gas. Data is sampled 5 times for each channel to obtain 5 quantized values. The data is ouput voltages of the sensor array.

TABLE 4
Quanitized values for toluene gas

| Time | CH 1 | CH 2 | CH 3 | CH 4 | CH 5 | CH 6 | CH 7 | CH 8 |
|---|---|---|---|---|---|---|---|---|
| 1 | −0.273 | −0.044 | −0.787 | −0.365 | −0.845 | −0.928 | −0.313 | −0.746 |
| 2 | 0.151 | 2.427 | −0.675 | 1.708 | −0.802 | 0.301 | −0.280 | 1.814 |
| 3 | 0.370 | 2.678 | −0.673 | 1.820 | −0.770 | 1.217 | −0.250 | 3.249 |
| 4 | 0.508 | 2.778 | −0.667 | 1.869 | −0.740 | 1.675 | −0.224 | 3.771 |
| 5 | 0.624 | 2.821 | −0.666 | 1.895 | −0.736 | 1.916 | −0.204 | 3.988 |

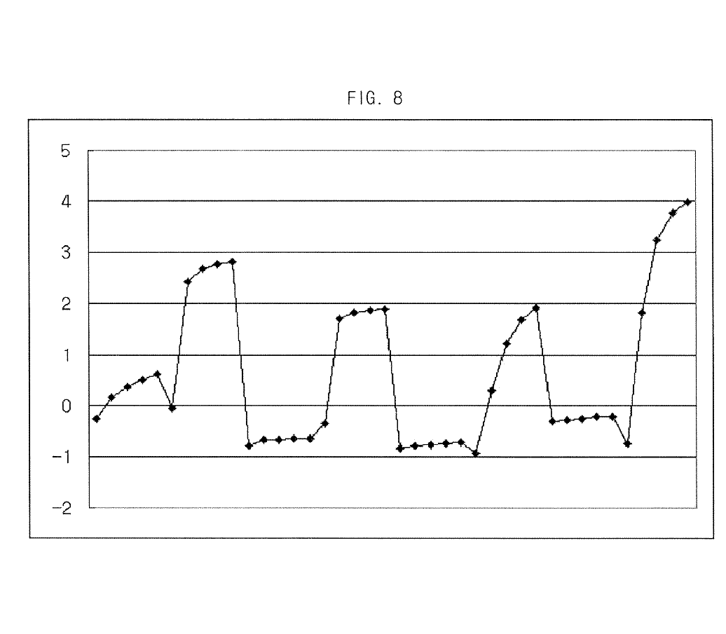

The values in Table 4 can be represented in a graph as shown in FIG. 8. The angle listed in Table 5, which is an angle of transition vector to x-axis, corresponds with the slope of broken lines in FIG. 8.

TABLE 5
Angles of transition vectors

| Value | Angle(°) | Value | Angle(°) | Value | Angle(°) | Value | Angle(°) |
|---|---|---|---|---|---|---|---|
| −0.273 | 22.98 | −0.787 | 6.39 | −0.845 | 2.46 | −0.313 | 1.89 |
| 0.151 | 12.35 | −0.675 | 0.11 | −0.802 | 1.83 | −0.28 | 1.72 |
| 0.37 | 7.86 | −0.673 | 0.34 | −0.77 | 1.72 | −0.25 | 1.49 |
| 0.508 | 6.62 | −0.667 | 0.06 | −0.74 | 0.23 | −0.224 | 1.15 |
| 0.624 | −33.74 | −0.666 | 16.75 | −0.736 | −10.87 | −0.204 | −28.46 |
| −0.044 | 67.97 | −0.365 | 64.25 | −0.928 | 50.87 | −0.746 | 68.66 |
| 2.427 | 14.09 | 1.708 | 6.39 | 0.301 | 42.49 | 1.814 | 55.13 |
| 2.678 | 5.71 | 1.82 | 2.81 | 1.217 | 24.61 | 3.249 | 27.56 |
| 2.778 | 2.46 | 1.869 | 1.49 | 1.675 | 13.55 | 3.771 | 12.24 |
| 2.821 | −74.51 | 1.895 | −69.95 | 1.916 | −65.84 | 3.988 | — |

The range from

$$- \frac{π}{2} to \frac{π}{2}$$

is divided into the number of states of the state transition models. In this embodiment, the number of states, n, is 10.

TABLE 6
Angle ranges of each state when n = 10

| State | Range |
|---|---|
| 1 | $0 \leq {S}_{1} < \frac{π}{10}$ |
| 2 | $\frac{π}{10} \leq {S}_{2} < \frac{π}{6}$ |
| 3 | $\frac{π}{6} \leq {S}_{3} < \frac{3 π}{10}$ |
| 4 | $\frac{3 π}{10} \leq {S}_{4} < \frac{2 π}{6}$ |
| 5 | $\frac{2 π}{6} \leq {S}_{6} < \frac{π}{2}$ |
| 6 | $- \frac{π}{10} \leq {S}_{6} < 0$ |
| 7 | $- \frac{π}{10} \leq {S}_{7} < - \frac{π}{10}$ |
| 8 | $- \frac{3 π}{10} \leq {S}_{8} < - \frac{π}{6}$ |
| 9 | $- \frac{2 π}{6} \leq {S}_{9} < - \frac{3 π}{10}$ |
| 10 | $- \frac{π}{2} \leq {S}_{10} < - \frac{2 π}{6}$ |

TABLE 7
State of each point

| Value | State | Value | State | Value | State | Value | State |
|---|---|---|---|---|---|---|---|
| −0.273 | 2 | −0.787 | 1 | −0.845 | 1 | −0.313 | 1 |
| 0.151 | 1 | −0.675 | 1 | −0.802 | 1 | −0.28 | 1 |
| 0.37 | 1 | −0.673 | 1 | −0.77 | 1 | −0.25 | 1 |
| 0.508 | 1 | −0.667 | 1 | −0.74 | 1 | −0.224 | 1 |
| 0.624 | 7 | −0.666 | 1 | −0.736 | 6 | −0.204 | 7 |
| −0.044 | 4 | −0.365 | 4 | −0.928 | 3 | −0.746 | 4 |
| 2.427 | 1 | 1.708 | 1 | 0.301 | 3 | 1.814 | 4 |
| 2.678 | 1 | 1.82 | 1 | 1.217 | 2 | 3.249 | 2 |
| 2.778 | 1 | 1.869 | 1 | 1.675 | 1 | 3.771 | 1 |
| 2.821 | 10 | 1.895 | 9 | 1.916 | 9 | 3.988 | — |

State number of each angle of Table 5 can be obtained by consulting Table 6, and is listed in Table 7.

The state number can be represented as a state sequence as shown in Table 8.

TABLE 8
State sequence of toluene gas

2 → 1 → 1 → 1 → 7 → 4 → 1 → 1 → 1 → 10 → 1 → 1 → 1 [symbol]
→ 1 → 1 → 4 → 1 → 1 → 1 → 9 → 1 → 1 → 1 → 1 → 6 → 3 [symbol]
→ 3 → 2 → 1 → 9 → 1 → 1 → 1 → 1 → 7 → 4 → 4 → 2 → 1

[symbol]

The data in Table 4 is converted into the state sequence through the ADSTM process.

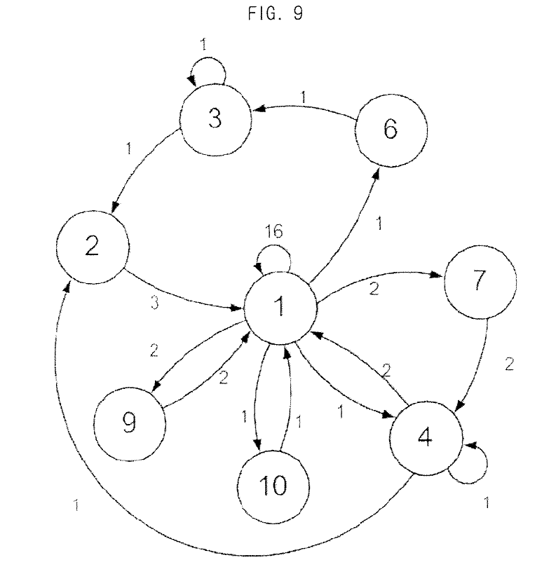

FIG. 9 is a diagram showing state transition between states. The numbers in FIG. 9 indicate the number of state transitions.

Table 9 a matrix corresponding to the state transition diagram of FIG. 9.

TABLE 9
State transition matrix of FIG. 9

| [symbol] | S₁ [symbol] | S₂ [symbol] | S₃ [symbol] | S₄ [symbol] | S₅ [symbol] | S₆ [symbol] | S₇ [symbol] | S₈ [symbol] | S₉ [symbol] | S₁₀ [symbol] |
|---|---|---|---|---|---|---|---|---|---|---|
| S₁ [symbol] | 16 [symbol] | 0 [symbol] | 0 [symbol] | 1 [symbol] | 0 [symbol] | 1 [symbol] | 2 [symbol] | 0 [symbol] | 2 [symbol] | 1 [symbol] |
| S₂ [symbol] | 3 [symbol] | 0 [symbol] | 0 [symbol] | 0 [symbol] | 0 [symbol] | 0 [symbol] | 0 [symbol] | 0 [symbol] | 0 [symbol] | 0 [symbol] |
| S₃ [symbol] | 0 [symbol] | 1 [symbol] | 1 [symbol] | 0 [symbol] | 0 [symbol] | 0 [symbol] | 0 [symbol] | 0 [symbol] | 0 [symbol] | 0 [symbol] |
| S₄ [symbol] | 2 [symbol] | 1 [symbol] | 0 [symbol] | 1 [symbol] | 0 [symbol] | 0 [symbol] | 0 [symbol] | 0 [symbol] | 0 [symbol] | 0 [symbol] |
| S₅ [symbol] | 0 [symbol] | 0 [symbol] | 0 [symbol] | 0 [symbol] | 0 [symbol] | 0 [symbol] | 0 [symbol] | 0 [symbol] | 0 [symbol] | 0 [symbol] |
| S₆ [symbol] | 0 [symbol] | 0 [symbol] | 1 [symbol] | 0 [symbol] | 0 [symbol] | 0 [symbol] | 0 [symbol] | 0 [symbol] | 0 [symbol] | 0 [symbol] |
| S₇ [symbol] | 0 [symbol] | 0 [symbol] | 0 [symbol] | 2 [symbol] | 0 [symbol] | 0 [symbol] | 0 [symbol] | 0 [symbol] | 0 [symbol] | 0 [symbol] |
| S₈ [symbol] | 0 [symbol] | 0 [symbol] | 0 [symbol] | 0 [symbol] | 0 [symbol] | 0 [symbol] | 0 [symbol] | 0 [symbol] | 0 [symbol] | 0 [symbol] |
| S₉ [symbol] | 2 [symbol] | 0 [symbol] | 0 [symbol] | 0 [symbol] | 0 [symbol] | 0 [symbol] | 0 [symbol] | 0 [symbol] | 0 [symbol] | 0 [symbol] |
| S₁₀ [symbol] | 1 [symbol] | 0 [symbol] | 0 [symbol] | 0 [symbol] | 0 [symbol] | 0 [symbol] | 0 [symbol] | 0 [symbol] | 0 [symbol] | 0 [symbol] |

Each different gas creates a different state transition matrix. However, even though the number of states or the sampling frequency is changed, but the state transition matrix remains similar when the gas is the same. Therefore, the state transition matrix can be used for analyzing the gas signal pattern.

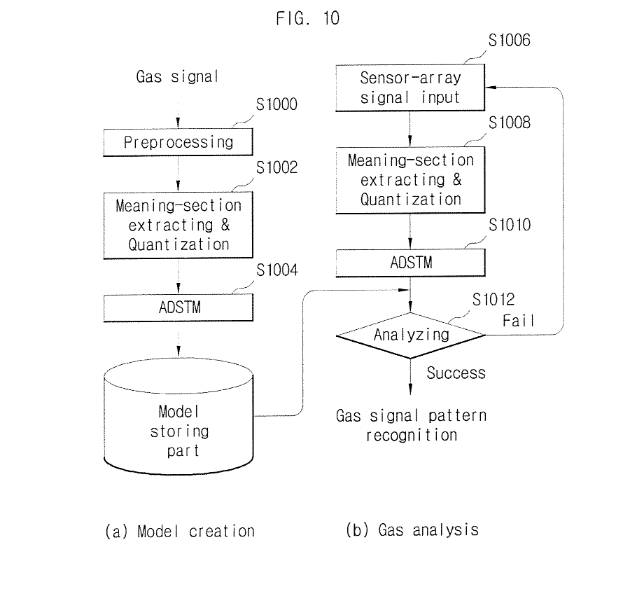

FIG. 10 is a gas recognition process diagram. Referring to FIG. 10, the gas recognition process according to the present invention is composed of two parts.

One part, which is called repository creation process, includes measuring a gas, creating a model for the gas by using the state transition model and storing the model in a repository. The other part, which is called gas recognition process, includes measuring an inputted gas, creating a model for the gas and analyzing the model by comparing it with the models in the repository.

When the same gas is repeatedly measured, data inputted from the sensor array shows a similar pattern as illustrated in FIG. 2. For example, FIG. 2 demonstrates six similar patterns that are results of measuring 2000 ppm of the toluene gas six times.

Since different gases have each different pattern, it is important to create a model reflecting unique feature of the gas in order to improve the analysis.

At step 1000, the values of gas signal measured by the sensor array 102 are preprocessed. As described earlier, during the preprocess procedure, average values representing the gas signal pattern are extracted from the values measured by the sensor array.

At step 1002, after preprocessing, meaning section is extracted and quantized. Here, the meaning section refers to the rising portion in the graph of the gas signal pattern, and the quantization is performed for a state transition modeling.

At step 1004, a state transition model is created based on the angle of the transition vectors. In this step, a state transition sequence is generated by using the angle between the transition vector and x-axis, whereupon a state transition matrix is created as a state transition model.

The state transition model is stored in the model storing part 114.

At step 1006, a new gas is measured by the sensor array. And at step 1008, a meaning section is extracted from the measured values of the new gas and is quantized.

At step 1010, the ADSTM procedure is processed for the new gas to create a state transition matrix, and at step 1012, this state transition matrix for the new gas is compared with the state transition matrices stored in the model storing part 114.

The state transition matrix shows how many times transitions occurred between states. For example, the cell (0, 0) tells that the transitions occurred twice from S₁ to S₁. Each gas has a different state transition matrix, so that a gas can be recognized by comparing its own state transition matrix with the stored transition matrices.

TABLE 10
State sequence
1 → 2 → 1 → 1 → 4 → 5 → 1 → 1 → 3 → 3 → 2

[symbol]

TABLE 11
State transition matrix before conversion

| [symbol] | S₁ [symbol] | S₂ [symbol] | S₃ [symbol] | S₄ [symbol] | S₅ [symbol] |
|---|---|---|---|---|---|
| S₁ [symbol] | 2 [symbol] | 1 [symbol] | 1 [symbol] | 1 [symbol] | 0 [symbol] |
| S₂ [symbol] | 1 [symbol] | 0 [symbol] | 0 [symbol] | 0 [symbol] | 0 [symbol] |
| S₃ [symbol] | 0 [symbol] | 1 [symbol] | 1 [symbol] | 0 [symbol] | 0 [symbol] |
| S₄ [symbol] | 0 [symbol] | 0 [symbol] | 0 [symbol] | 0 [symbol] | 1 [symbol] |
| S₅ [symbol] | 1 [symbol] | 0 [symbol] | 0 [symbol] | 0 [symbol] | 0 [symbol] |

TABLE 12
State transition matrix after conversion

| [symbol] | S₁ [symbol] | S₂ [symbol] | S₃ [symbol] | S₄ [symbol] | S₅ [symbol] |
|---|---|---|---|---|---|
| S₁ [symbol] | 1 [symbol] | 1 [symbol] | 1 [symbol] | 1 [symbol] | 0 [symbol] |
| S₂ [symbol] | 1 [symbol] | 0 [symbol] | 0 [symbol] | 0 [symbol] | 0 [symbol] |
| S₃ [symbol] | 0 [symbol] | 1 [symbol] | 1 [symbol] | 0 [symbol] | 0 [symbol] |
| S₄ [symbol] | 0 [symbol] | 0 [symbol] | 0 [symbol] | 0 [symbol] | 1 [symbol] |
| S₅ [symbol] | 1 [symbol] | 0 [symbol] | 0 [symbol] | 0 [symbol] | 0 [symbol] |

An experiment was conducted according to the present invention. In this experiment, eight different gases were measured to create eight different gas models with an eight-channel gas sensor array.

When an object gas is measured, a state transition matrix of the object gas is generated to compare with the state transition matrices of the 8 different gases. The comparision is performed as follows:

The state sequence in Table 10 is converted to a state transition matrix in Table 11.

The matrix in Table 11 can be converted into the matrix in Table 12 by assigning 1 to a cell where a state transition occurs, and assigning 0 to a cell where no state transition occurs.

And, it is assumed here that the state transition matrix for a new gas is O and the state transition matrix stored in the model storing part 114 is M.

Accordingly, similarity between these two matrices can be computed by Mathmatical Formula 5. Here, n(O) or n(M) is defined as the number of elements of which value is 1.

$$similarity = \frac{n ( O ⋂ M )}{n ( O ⋃ M )} \qquad Mathematical Formula 4$$

Here, n(O∩M) means the number of cells, both of which have a value of 1 and are in the same column and row, in either matrix.

Also, n(O∪ M) means the number of cells, either of which has a value of 1 and are in the same column and row, in either matrix.

The above Formula is a useful means to check relative similiarity.

A similarity threshold is determined based on experimental data of eight different gases that are obtained through a number of measurements.

Here, the threshold similarity refers to a minimum value among a number of the similarities that are calculated for the same.

In the case that a similarity for a new gas does not exceed the threshold, the similarity check is repeated a predetermined number of times.

If the maximum similarity value among the similarity values obtained through the repeated similarity checks is smaller than the threshold, then the new gas is determined to be unrecognizable.

The following describes a method verifying the present invention.

In this experiment, eight different hydrocarbon based gases, as listed in Table 14, were detected by a sensor array through eight different channels. Those eight gases are toluene, benzene, ethanol, methanol, 2-propanol, n-hexane, n-heptane and cyclohexane, and were measured under the conditions shown in Table 14.

TABLE 13
Organization of sensor array

| | | | R_vac | R_m1 |
|---|---|---|---|---|
| Channel | Sensor | | (B/A, kΩ) | (B/A, kΩ) |
| CH1 | CBC#1 | EC_THF_2000 | 15//3.3 | 3.1//3.1 |
| CH2 | CBC#2 | HPC_DGD_Ch_2000 | 69//48 | 47//47 |
| CH3 | CBC#5 | PVS_THF_2000 | 855//25 | 23//23 |
| CH4 | CBC#7 | PVA + DGD_Ch_700 2nd | 125//79 | 79//79 |
| CH5 | CBC#10 | PS-PIP-PS_Ch_2000 | 22//21 | 18//19 |
| CH6 | CBC#12 | PVP + DOP_Ch_2000 | 64//56 | 59//67 |
| CH7 | CBC#14 | PS-BD_Ch_2000 | 3.9//9.6 | 9.7//9.7 |
| CH8 | CBC#26 | PEG_THF_2000 | 309//123 | 138//181 |

TABLE 14
Object gases and measurment conditions

| Gas | Temperature | ppm | Date |
|---|---|---|---|
| Toluene | 20 | 2000 | 2005. 2. 1 |
| Benzene | 20 | 5000 | 2005. 2. 1 |
| Ethanol | 20 | 5000 | 2005. 2. 2 |
| Methanol | 20 | 5000 | 2005. 2. 2 |
| 2-Propanol | 20 | 5000 | 2005. 2. 2 |
| n-Hexane | 20 | 5000 | 2005. 2. 2 |
| n-Heptane | 20 | 2500 | 2005. 2. 2 |
| Cyclohexane | 20 | 5000 | 2005. 2. 2 |

Data was collected through measuring the eight gases six times. Among six results, four random results were used to determine a threshold value, and the rest two results were used to verify the invention.

In order to obtain an optimal threshold value, the experiment is repeated by varying the number of states n and the sampling frequency k, which are variables of the modeling method of the invention.

The eight gases are analyzed by checking similarity with seven models among eight gas models.

A first experiment was conducted by fixing the sampling frequency k to 13 but varying the number of state transitions n. Under this condition, the number of quantization elements is 104 (m=104) since the number of the channels is 8.

Also, by varying the number of states from 20, 60 to 100, analysis rate and accuracy can be compared, so that the threshold value can be speculated.

A second experiment was conducted by varying the sampling frequency from 7, 13, to 25 but fixing the number of states to 20.

This experiment aims to see the influence of regulating sampling interval on the model and the threshold value.

Next, a new gas is analyzed by using threshold value determined through the above experiment.

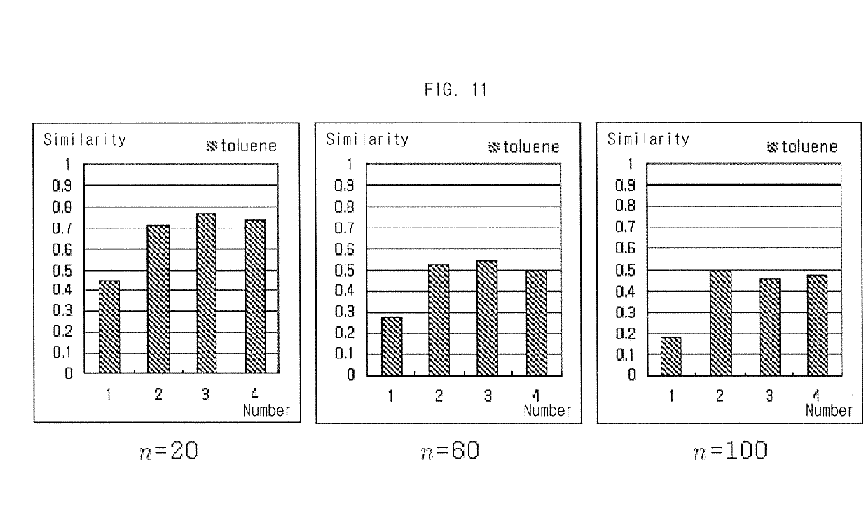

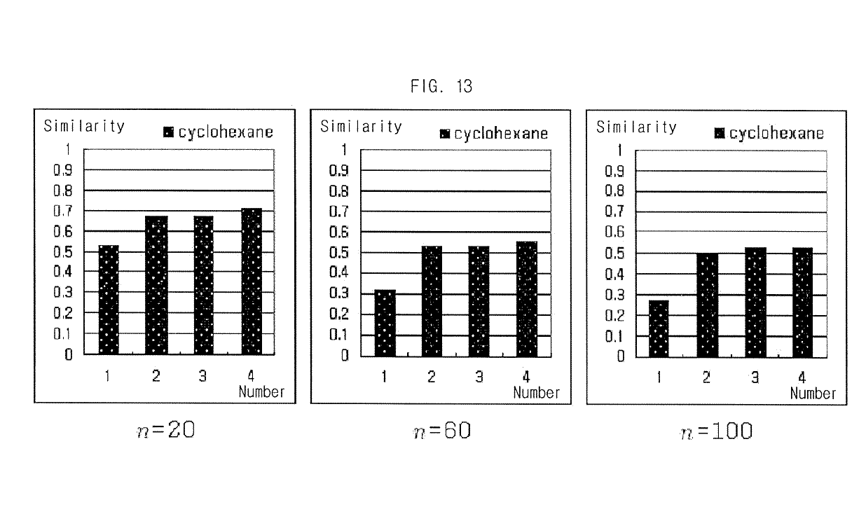

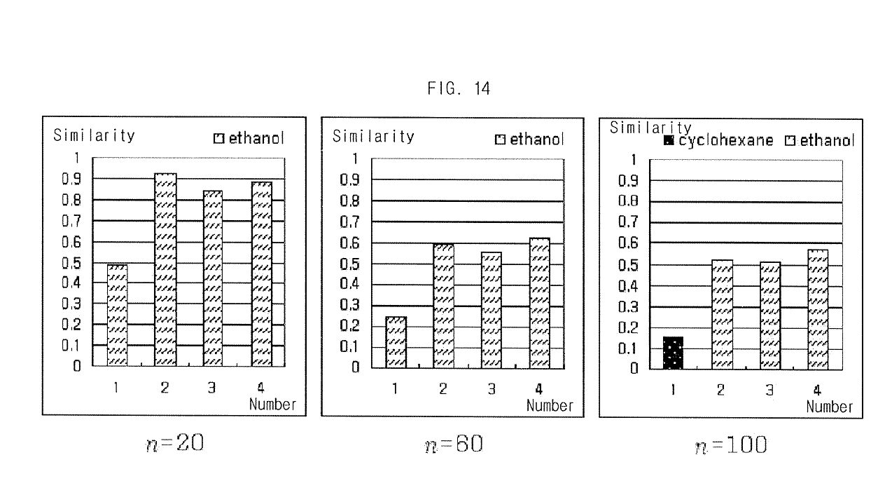

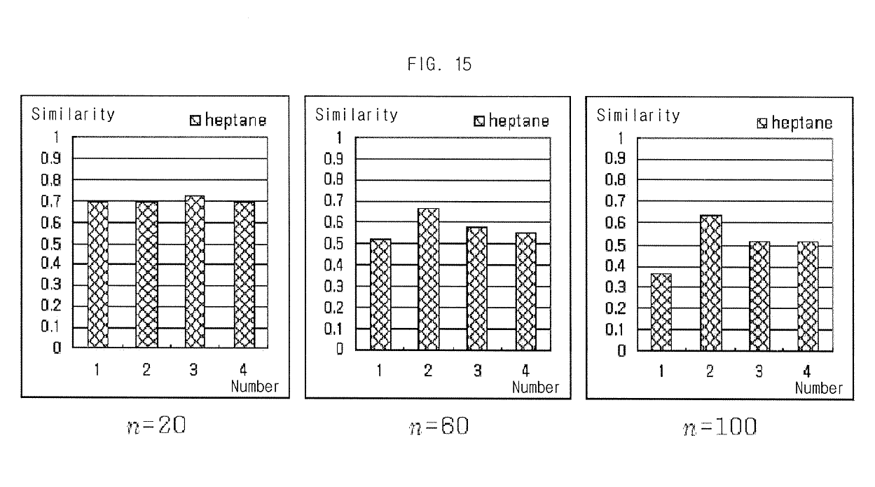

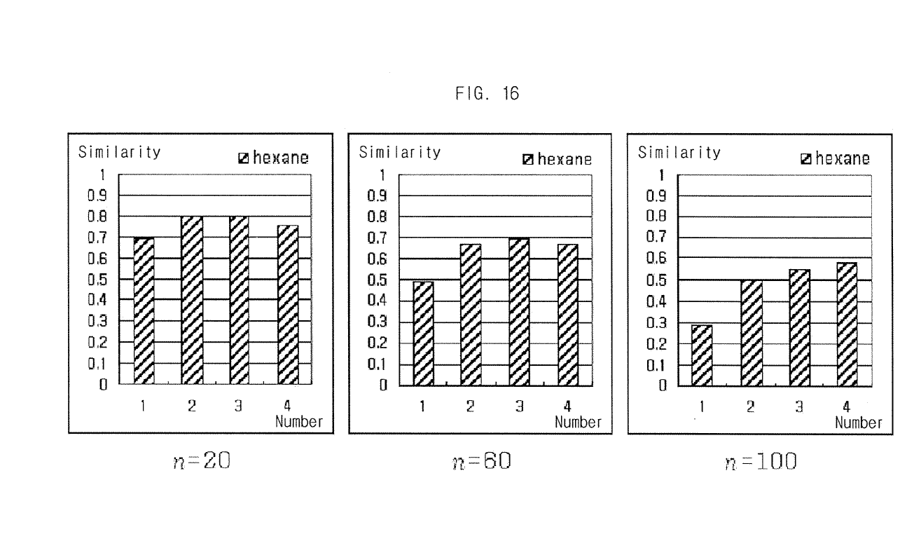

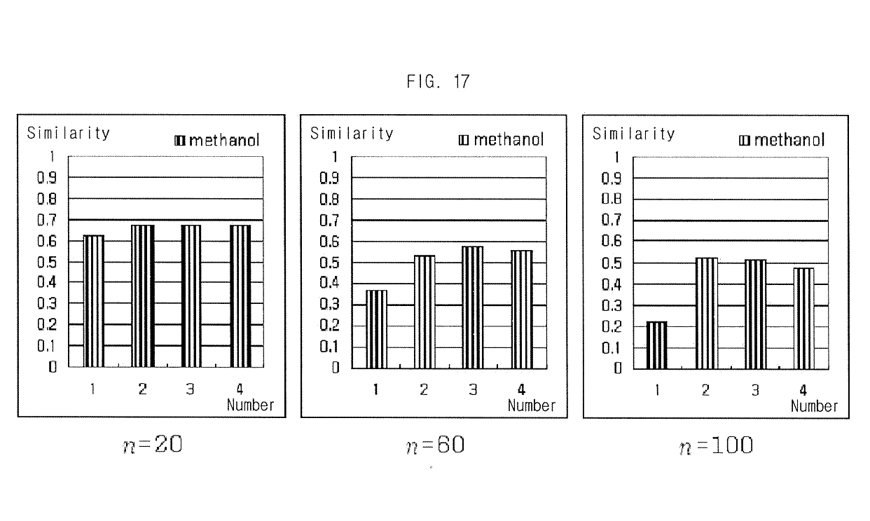

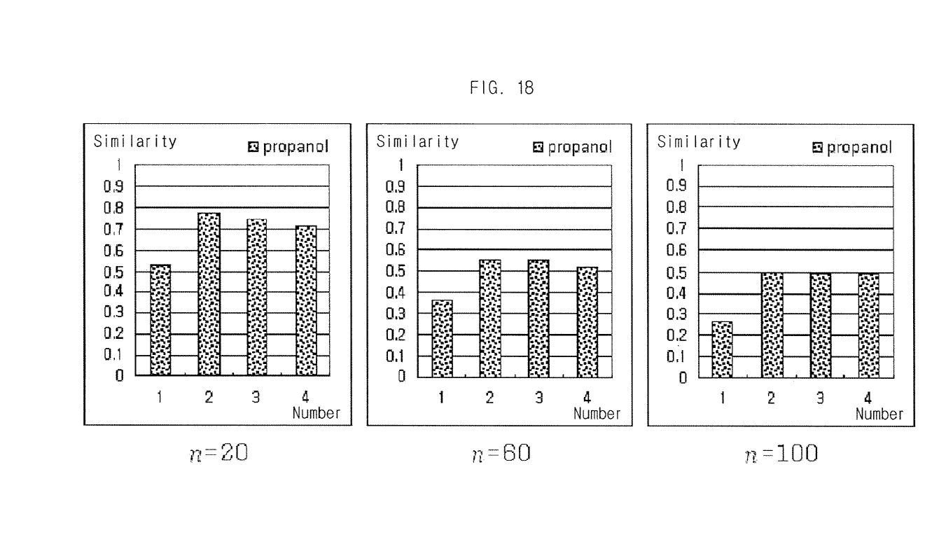

FIGS. 11 to 18 are experimential result of a case where a benzene model was removed from the model repository and the number of states varies from 20, 60 to 100.

It can be known that as the number of states n becomes larger, the similarity decreases.

The process of converting the values measured by the sensors into the state transition model has time complexity O(N) so that almost equal time is spent for creating each model.

However, analyzing has time complexity O(N²), so that analysis time increases by square times of the ratio at which the number of states is increased.

In case of detecting gas leakeage, analysis time is an important factor. Accordingly, it can be concluded that the number of states should be chosen among small numbers that can represent the characteristic of gas pattern.

There are two important points to consider when determining the threshold value for the gas recognition.

One is that when detecting an unknown gas, the threshold value should be larger than the similarity value to the unknown gas.

The other is that the threshold value should be the same as or smaller than the similarity value to the model gas.

For example, when the number of states is 20, the threshold value can be determined using the experimental results in FIGS. 11 through 18.

Since no benzene model is stored in the model repository, benzene gas should not be distinguished.

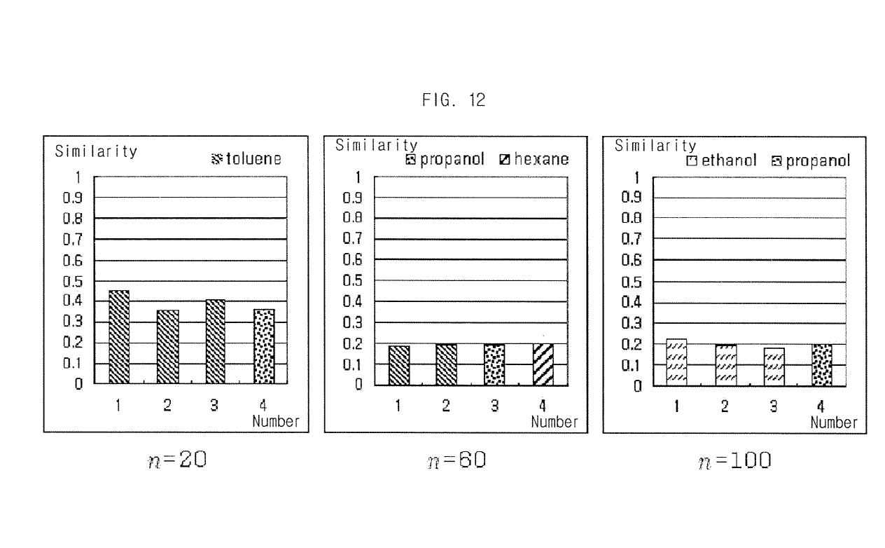

Referring to FIG. 12, the maximum similarity value of the benzene gas is 0.446809, which is a similarity value to the toluene model.

Therefore, in order to prevent benzene from misrecognized as toluene, the threshold value should be lager than 0.446809.

Also, the threshold value should be equal with or smaller than a smallest similarity value among the similarity values of the gases except from the benzene, which is 0.484848 in this experiment.

Therefore, the range of the threshold value can be determined as the Mathmatical Formula 5.

0.446809<threshold≦0.484848 Mathmatical Formula 5

By setting a value within this range as the threshold value, a gas can be recognized through a small number of measurements.

Therefore, the threshold values for seven gas models except for the benzene model are chosen within this range.

In order to decide a threshold value with respect to all the gas models, a largest similarity value should be known among the similarity values of all the cases where one of the eight gases is missed among the eight gas models.

Table 15 shows similarity values of a gas of which gas model is removed from the model repository to the rest seven models under the condition that n=20.

Referring to Table 15, in most cases, the similarity value of a gas decreases when the gas model repository contains no gas model of the same gas.

However, when the gas model for heptane or hexane is removed, the largest threshold value increases, causing an error in recognizing gas.

Therefore, additional process is needed to lower the largest threshold value.

TABLE 15
Similarity values of a gas of which gas model is removed

| | Toluene | | Benzene | | Cyclohexane | |
|---|---|---|---|---|---|---|
| No. | Nearest gas | Similarity | Nearest gas | Similarity | Nearest gas | Similarity |
| 1 | cyclohexane | 0.318182 | toluene | 0.446809 | propanol | 0.444444 |
| 2 | cyclohexane | 0.348837 | toluene | 0.354167 | propanol | 0.444444 |
| 3 | benzene | 0.375000 | toluene | 0.408163 | heptane | 0.470588 |
| 4 | benzene | 0.381818 | propanol | 0.363636 | heptane | 0.457143 |
| | Ethanol | | Heptane | | Hexane | |
| No. | Nearest gas | Similarity | Nearest gas | Similarity | Nearest gas | Similarity |
| 1 | heptane | 0.300000 | hexane | 0.666667 | heptane | 0.548387 |
| 2 | propanol | 0.309524 | hexane | 0.620690 | heptane | 0.633333 |
| 3 | propanol | 0.325000 | hexane | 0.533333 | heptane | 0.633333 |
| 4 | propanol | 0.317073 | hexane | 0.666667 | heptane | 0.548387 |

| | |
|---|---|
| Methanol | Propanol |

| No. | Nearest gas | Similarity | Nearest gas | Similarity |
|---|---|---|---|---|
| 1 | ethanol | 0.270270 | cyclohexane | 0.341463 |
| 2 | ethanol | 0.270270 | cyclohexane | 0.341463 |
| 3 | ethanol | 0.270270 | hexane | 0.371429 |
| 4 | heptane | 0.282051 | hexane | 0.382353 |

In the foregoing experiment, the number of states was varied while the sampling frequency was fixed. An optimal result was produced when n=20.

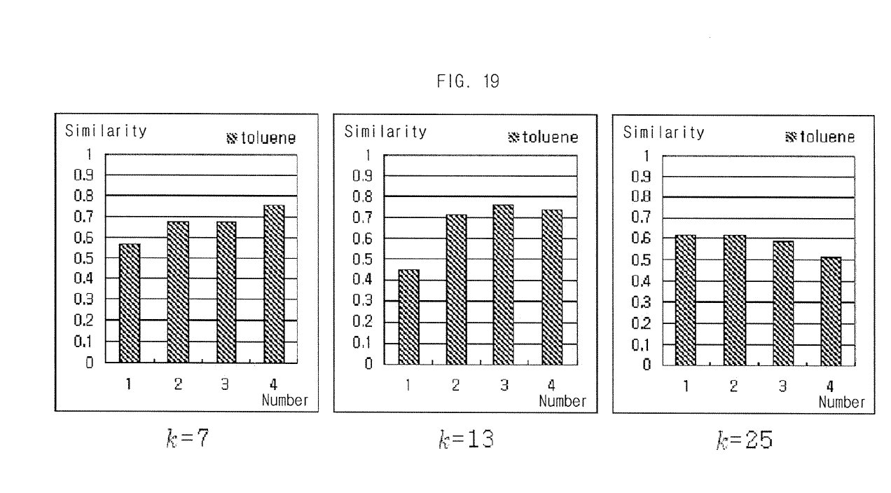

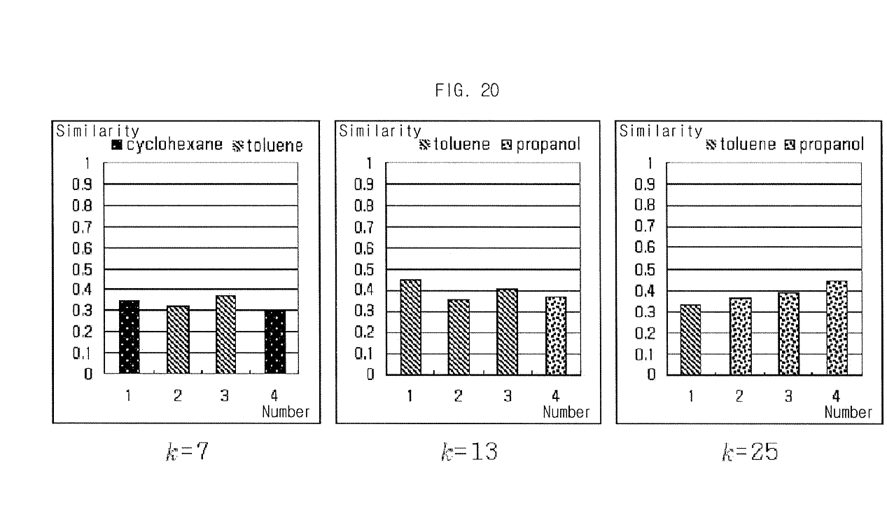

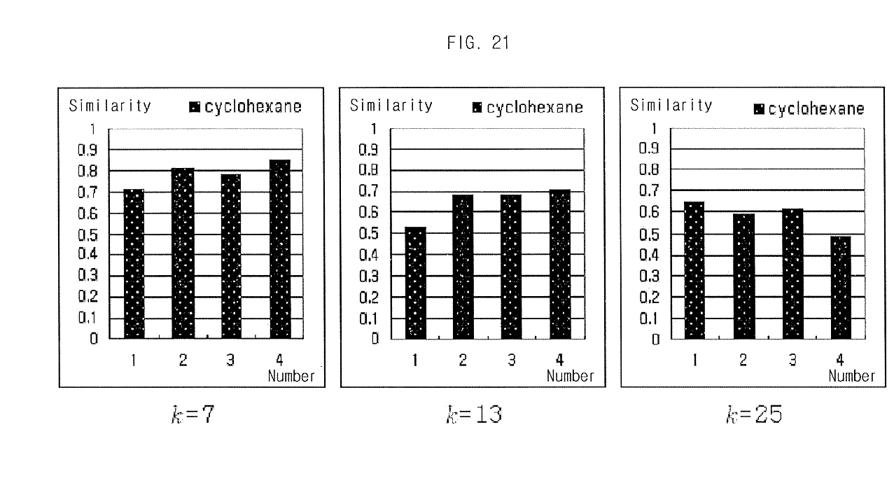

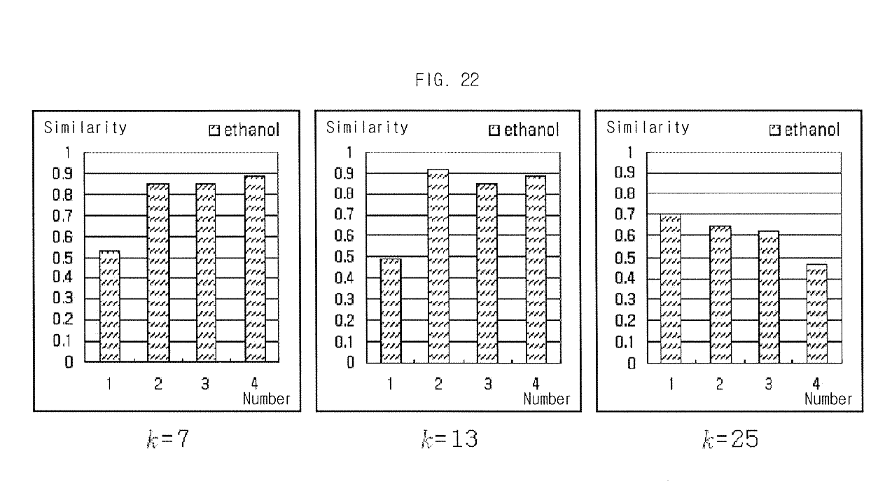

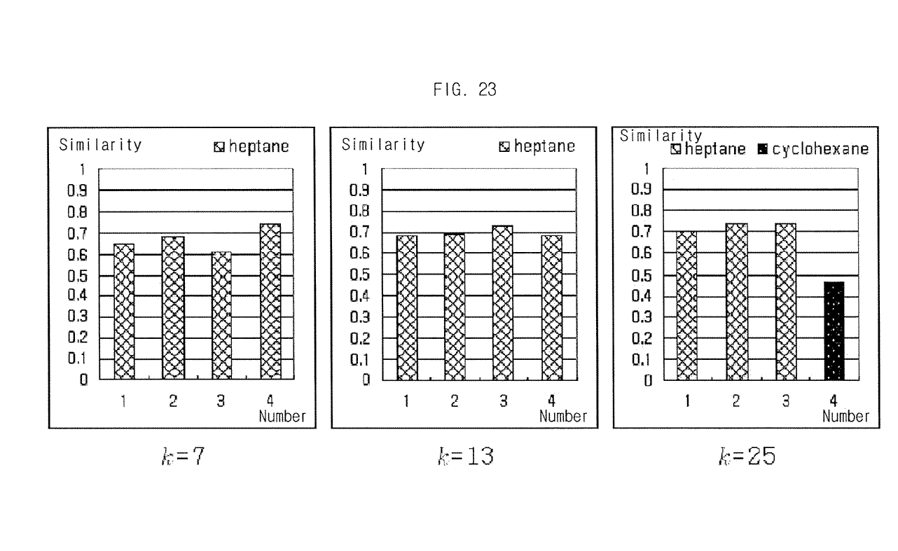

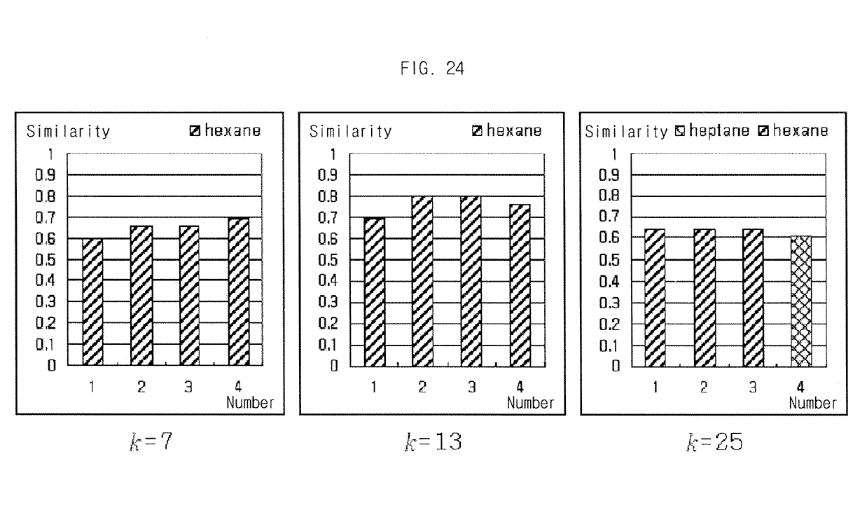

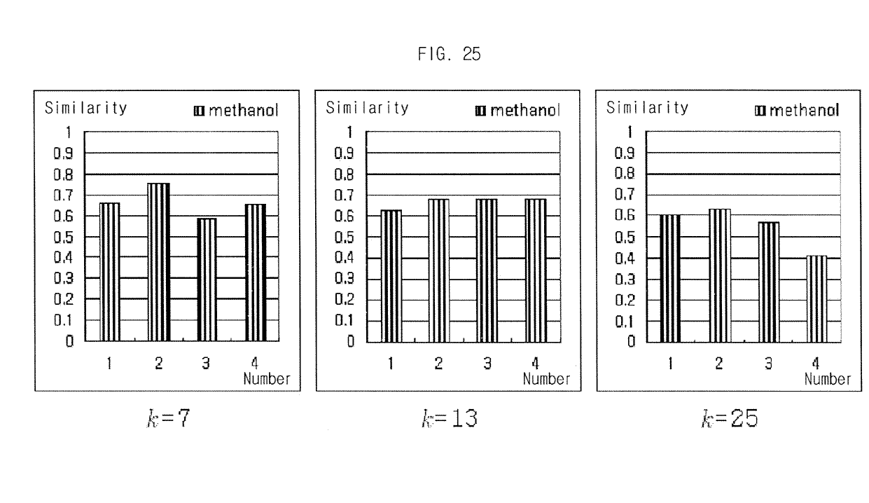

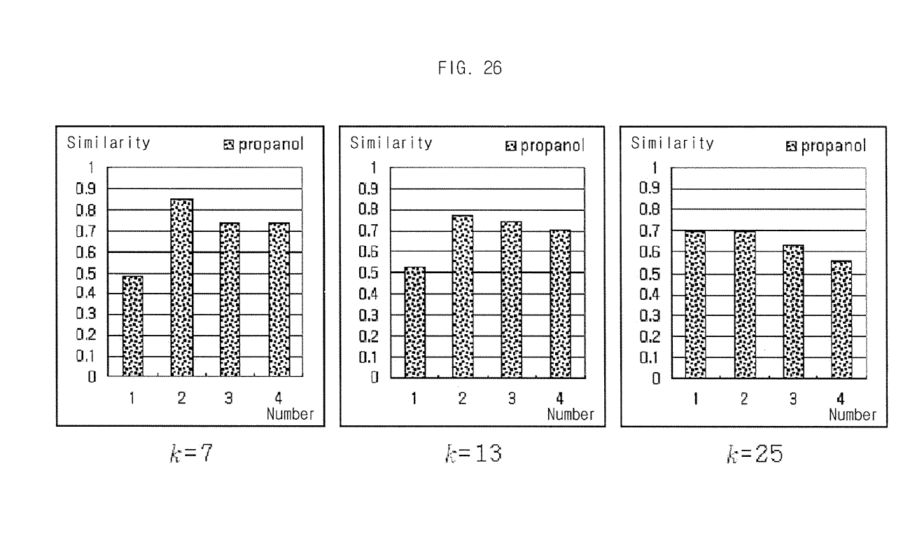

Another experiment was conducted under the condition that n is fixed to 20, while the sampling frequency is varied from 7, 13 to 25. FIGS. 19 through 26 show the result of the case where the benzene model is removed from the gas model repository.

Referring to FIGS. 19 through 26, similarity values increases or remains the same when the sampling frequency k is increased.

For instance, as shown in Table 16 are similarity values of a gas of which gas model is removed under the condition that k=7.

Comparing Table 17 and Table 18, it can be seen that the similarity values of the heptane and the hexane gases that were considerably large under the condition that k=13, are lowered to an appropriate level under the condition that k=7.

TABLE 16
Similarity values of a gas of which gas model is removed

| | Toluene | | Benzene | | Cyclohexane | |
|---|---|---|---|---|---|---|
| No. | Nearest gas | Similarity | Nearest gas | Similarity | Nearest gas | Similarity |
| 1 | cyclohexane | 0.272727 | cyclohexane | 0.350000 | heptane | 0.405405 |
| 2 | benzene | 0.357143 | toluene | 0.319149 | heptane | 0.432432 |
| 3 | cyclohexane | 0.333333 | toluene | 0.377778 | heptane | 0.368421 |
| 4 | benzene | 0.363636 | cyclohexane | 0.300000 | heptane | 0.384615 |
| | Ethanol | | Heptane | | Hexane | |
| No. | Nearest gas | Similarity | Nearest gas | Similarity | Nearest gas | Similarity |
| 1 | cyclohexane | 0.324324 | hexane | 0.441176 | heptane | 0.470588 |
| 2 | hexane | 0.333333 | hexane | 0.470588 | heptane | 0.515152 |
| 3 | methanol | 0.300000 | hexane | 0.454545 | heptane | 0.515152 |
| 4 | hexane | 0.325000 | hexane | 0.529412 | heptane | 0.416667 |

| | |
|---|---|
| Methanol | Propanol |

| No. | Nearest gas | Similarity | Nearest gas | Similarity |
|---|---|---|---|---|
| 1 | propanol | 0.333333 | methanol | 0.342105 |
| 2 | propanol | 0.361111 | ethanol | 0.351351 |
| 3 | propanol | 0.222222 | methanol | 0.289474 |
| 4 | propanol | 0.393939 | methanol | 0.289474 |

TABLE 17
Similarity values of heptane and hexane when k = 13

| | |
|---|---|
| Heptane | Hexane |

| Nearest gas | Similarity | Nearest gas | Similarity |
|---|---|---|---|
| hexane | 0.666667 | heptane | 0.548387 |
| hexane | 0.620690 | heptane | 0.633333 |
| hexane | 0.533333 | heptane | 0.633333 |
| hexane | 0.666667 | heptane | 0.548387 |

TABLE 18
Similarity values of heptane and hexane when k = 7

| | |
|---|---|
| Heptane | Hexane |

| Nearest gas | Similarity | Nearest gas | Similarity |
|---|---|---|---|
| hexane | 0.441176 | heptane | 0.470588 |
| hexane | 0.470588 | heptane | 0.515152 |
| hexane | 0.454545 | heptane | 0.515152 |
| hexane | 0.529412 | heptane | 0.416667 |

Based on the above experiments, it can be concluded that an optimal result can be obtained when k=7 and n=20.

Such a result tells that reducing sampling interval (in other words, increasing the sampling frequency) deteriorates the features of gas signal pattern.

Here, the largest value in Table 16, which is 0.529412, can be determined as a threshold value.

Aother gas was tested twice under the same condition, which verified the reliability of this threshold value, 0.529412.

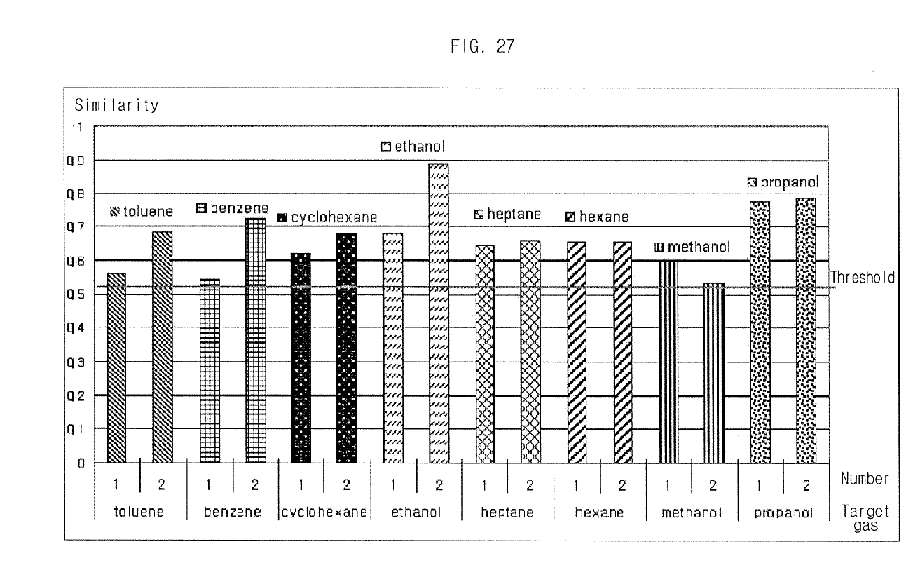

As shown in FIG. 27, all the gases can be distinguished by setting the threshold value smaller than the similarity values of all the gases.

Through the tests as described above, it is confirmed that the modeling method according to the present invention can be applied to recognizing signal pattern and also to distinguishing the gases.

Also, by using state transition model, this modeling method requires less resource and time.

While the invention has been described with reference to the disclosed embodiments, it is to be appreciated that those skilled in the art can change or modify the embodiments without departing from the scope and spirit of the invention or its equivalents as stated below in the claims.

What is claimed is:

1. A method for analyzing a signal pattern detected by a sensor array that comprises one or more gas sensors, the method comprising:
converting multidimensional data outputted from the sensor array to linear data, the data containing information on one or more reference gases;
creating an ADSTM(Angle Difference-based State Transition Model) by using the converted data; and
analyzing a gas by using the ADSTM when the sensor array outputs data of the gas.

2. The method of claim 1, wherein data converting step further comprises:
preprocessing the multidimensional data;
extracting a meaning section from the preprocessed data;
linearizing data contained in the meaning section;
extracting a k numbe of samples from the linearized data (k is a natural number); and
quantizing the data samples.

3. The method of claim 2, wherein the preprocessing step further comprises excluding data having a value deviated from an average value out of the multidimensional data.

4. The method of claim 2, wherein the multidimensional data repeats increasing and decreasing, and the extracting a meaning section step further comprises extracting a portion from where the multidimensional data begins to increase to where the multidimensional data reaches a peak.

5. The method of claim 2, wherein the linearizing data contained in the meaning section further comprises converting the extracted multidimensional data of the meaning section into linear data in correspondence with the order of the one or more sensors.

6. The method of claim 2, wherein the creating a state transition model further comprises:
generating a plurality of transition vectors using the quantized data;
dividing a predetermined section into an n number of sections, each section defined as one state;
generating a state sequence by corresponding angles of the transition vectors with the n number of sections; and
generating for each reference gas one or more first state transition matrices to correspond to the number of transitions by using the state sequence.

7. The method of claim 6, wherein the generating a plurality of transition vectors further comprises generating the transition vector by using i th data and (i+1) th data of the quantized data (i is a natural number).

8. The method of claim 6, wherein the predetermined section has a range from −π/2 to π/2.

9. The method of claim 8, wherein each state has a range determined by the following formulas:
$$\begin{matrix}\frac{π}{n} \times ( i - 1 ) \leq {s}_{i} < \frac{π}{n} \times i & ( 0 < i \leq \frac{n}{2} ) \\ - \frac{π}{n} \times ( i - \frac{n}{2} ) \leq {s}_{i} < - \frac{π}{n} ( i - \frac{n}{2} + 1 ) & ( \frac{n}{2} < i \leq n )\end{matrix}$$

10. The method of claim 6, wherein the step of analyzing a gas further comprises generating a second state transition matrix for the gas, and calculating similarity of the second state transition matrix with respect to each first state transition matrix.

11. The method of claim 10, wherein the step of analyzing a gas further comprises analyzing the gas by using a threshold value predetermined based on the similarities.

12. The method of claim 11, wherein the threshold value is a smallest value among similarities of the first state transition matrix with respect to all of the reference gases.

13. A recorded medium readable by a computer, tangibly embodying a program of instructions executable by the computer to perform a method according to any of claims 1 to 12.

14. An apparatus for analyzing a signal pattern detected by a sensor array that comprising one or more gas sensors, the apparatus comprising:
a preprocessor that preprocesses multidimensional data outputted from the sensor array;
a meaning-section extractor that extracts a meaning section from the preprocessed data;
a linearizing part that linearizes the multisimensional data contained in the meaning section;
a quantizing part that extracts a k number of samples (k is a natural number) from the linearized data and quantizes the samples;
a model creating part that creates an ADSTM(Angle Difference-based State Transition Model) by using the quantized data; and
an analyzing part that analyzes a gas by using the ADSTM when the sensor array outputs data of the gas.

15. The apparatus of claim 14, wherein the preprocessor excludes data having a value deviated from an average value out of the multidimensional data.

16. The apparatus of claim 14, wherein the multidimensional data repeats increasing and decreasing, and the meaning-section extractor extracts a portion from where the multidimensional data begines to increase to where the multidimensional data reaches a peak.

17. The apparatus of claim 14, wherein the linearizing part converts the extracted multidimensional data of the meaning section into linear data in correspondence with the order of the one or more sensors.

18. The apparatus of claim 14, wherein the model creating part further comprises:
a transition vector generating part that generates a plurality of transition vectors by using the quantized data;
a section dividing part that divides a predetermined section into an n number of sections, each section defined as one state;
a state sequence generating part that generates a state sequence by corresponding angles of the transition vectors with the n number of sections; and
a state transition matrix generating part that generates for each reference gas one or more first state transition matrices to correspond to the number of transitions by using the state sequence.

19. The apparatus of claim 18, wherein the state transition vector generating part generates the transition vector by using i th data and (i+1) th data of the quantized data (i is a natural number).

20. The apparatus of claim 18, wherein the predetermined section has a range from −π/2 to π/2.

21. The apparatus of claim 20, wherein each state has a range determined by the following formulas:
$$\begin{matrix}\frac{π}{n} \times ( i - 1 ) \leq {s}_{i} < \frac{π}{n} \times i & ( 0 < i \leq \frac{n}{2} ) \\ - \frac{π}{n} \times ( i - \frac{n}{2} ) \leq {s}_{i} < - \frac{π}{n} ( i - \frac{n}{2} + 1 ) & ( \frac{n}{2} < i \leq n )\end{matrix}$$

22. The apparatus of claim 18, wherein the analyzing part generates a second state transition matrix for the gas and calculates similarity of the second state transition matrix with respect to each first state transition matrix.

23. The apparatus of claim 22, wherein the analyzing part analyzes the gas by using a threshold value predetermined based on the calculated similarities.

24. The apparatus of claim 23, wherein the threshold value is a smallest value among similarities of the first state transition matrix with respect to all of the reference gases.
